(19) United States
(12) Patent Application Publication
(10) Pub. No.: US 2025/0262443 A1
(43) Pub. Date: Aug. 21, 2025

(54) COMPUTER IMPLEMENTED METHOD AND SYSTEM FOR PROGRAMMING LEADLESS CARDIAC PACEMAKERS

(71) Applicant: BIOTRONIK SE & Co. KG, Berlin (DE)

(72) Inventors: Brian M. TAFF, Portland, OR (US); Kurt SWENSON, Corvallis, OR (US); Dirk MUESSIG, West Linn, OR (US); Hannes KRAETSCHMER, West Linn, OR (US); Dean BERGSTROM, West Linn, OR (US)

(73) Assignee: BIOTRONIK SE & Co. KG, Berlin (DE)

(21) Appl. No.: 18/857,245

(22) PCT Filed: Apr. 4, 2023
(86) PCT No.: PCT/EP2023/058807

§ 371 Date: Oct. 16, 2024

Related U.S. Application Data
(60) Provisional application No. 63/335,327, filed on Apr. 27, 2022.

(30) Foreign Application Priority Data
May 20, 2022 (EP) 22174486.5

(51) Int. Cl. A61N 1/372; A61N 1/02; A61N 1/37; A61N 1/375

(52) U.S. Cl. CPC A61N 1/37264; A61N 1/025; A61N 1/3708; A61N 1/37223; A61N 1/37247; A61N 1/37288; A61N 1/3756

(57) ABSTRACT

A computer implemented method for programming leadless cardiac pacemakers including assigning a unique identifier to each of the first leadless cardiac pacemaker and the second leadless cardiac pacemaker by means of the computing device, and preconfiguring the parameters of the first leadless cardiac pacemaker and/or the second leadless cardiac pacemaker by means of the computing device regardless of a location of the first leadless cardiac pacemaker and the second leadless cardiac pacemaker with respect to a transmit-receive unit of the computing device. Moreover, the invention relates to a system for programming leadless cardiac pacemakers. In addition, a computer program and a computer-readable data carrier are also disclosed.

### CROSS-REFERENCE TO RELATED APPLICATIONS

This application is the United States National Phase under 35 U.S.C. § 371 of PCT International Patent Application No. PCT/EP2023/058807, filed on Apr. 4, 2023, which claims the benefit of European Patent Application No. 22174486.5, filed on May 20, 2022 and U.S. Provisional Patent Application No. 63/335,327, filed on Apr. 27, 2022, the disclosures of which are hereby incorporated by reference herein in their entireties.

### TECHNICAL FIELD

The present invention relates to a computer implemented method for programming leadless cardiac pacemakers. Furthermore, the present invention relates to a system for programming leadless cardiac pacemakers.

### BACKGROUND

International Publication No. WO 2008/042610 A2 describes a programming device consisting of a manufacturer-specific telemetry module coupled to a computer.

European Patent No. EP 2 181 729 B1 discloses a universal programming device for patient-specific medical devices, such as implants. The programmer comprises an RF transceiver (transmitter/receiver), a control device, and a man-machine interface or a connector for the man-machine interface. The RF transceiver is configured to receive and transmit data in the MICS band. The control device is connected to the transceiver and has preconfigured software interfaces such that the programmer is expandable with control software modules, wherein the preconfigured software interfaces define a unified interface for controlling the transceiver that can be accessed by control software modules. The human-machine interface, for example, a keyboard and/or display or the connector for a human-machine interface, is connected to the control device.

In traditional IPG (Implantable Pulse Generator) systems a new implant for replacement is readily configured entirely independently from any old implant in need of replacement. Such interactions occur by establishing some form of ON state in the new implant that brings it out of its shipping configuration, often referred to as a shelf state. Depending on the manufacturer, this may be an implant-ready condition, e.g., auto-implant detection ready or a state where the implant has been programmed to a specific pacing mode with specified pace output conditions and becomes active, even without having any physical attachments to patient anatomy.

In these legacy pocket-based implant systems, the change-out can largely involve the clinician rapidly swapping the lead connections from the old device to the new device. This change-out does not administer therapy to new unknown tissue interfaces within the heart but instead borrows the same leaded connections that once relayed output from the old device and simply pipelines in input from the new device.

In leadless implants, added caretaking is needed as not only a new device is provisioning brady support to the heart, but it is doing so necessarily at a new physical interface even though it is in the same chamber as the old implant within the heart. The variable quality of implantation site interfacing with the heart's conduction system adds risk to the establishment of a reliable electrode engagement with the heart's electrical conduction system and, as a result, it merits specific management/handling to ensure robust/safe replacement therapy administration.

With traditional IPG systems an Elective Replacement Interval (ERI) is provided to indicate the time for replacement. ERI is typically designed to provide a low-power therapy for a period of time sufficient to be detected during the next follow-up. It is common to wait until ERI is detected before scheduling replacement. Follow-up intervals are typically 6 months so it is possible that patients will receive several months of reduced therapy and the old device will be replaced without completely depleting the battery first.

The traditional mechanism for controlling what implant a programmer is interacting with is by controlling what implant the inductive field from the programmer's wand covers. This has typically been restricted to a single active implant in the patient. When RF communications between the programmer and the implant made far-field communications possible, the standard became that near-field inductive communications needs to be used to enable this connection, with the connection still restricted to a single implant within the wand field.

There is currently no known manufacturer-based solution to facilitate a guided process for device replacement/change-out in leadless pacer systems. Instead, the programmer must interact with the old and new implants virtually independently to direct specific behaviors and program settings to each device.

The process largely leaves the clinician to “figure it out” based on past IPG clinical engagements. Unfortunately, this approach treats the end-of-service use case as one where two separate processes need to be handled to instate what ultimately amounts to a need for coordinated therapy administration.

The present disclosure is directed toward overcoming one or more of the above-mentioned problems, though not necessarily limited to embodiments that do.

### SUMMARY

It is therefore an object of the present invention to provide an improved method for programming leadless cardiac pacemakers, in which device replacement/change-out is orchestrated in a more coordinated, user friendly manner.

At least the object is solved by a computer implemented method for programming leadless cardiac pacemakers having the features of claim 1.

In addition, at least the object is solved by a system for programming leadless cardiac pacemakers having the features of claim 13, a computer program of claim 14 and the computer-readable data carrier of claim 15. Further developments and advantageous embodiments are defined in the dependent claims.

The present invention provides a computer implemented method for programming leadless cardiac pacemakers. The method comprises providing a computing device, in particular a programmer, configured to set a plurality of parameters of at least a first leadless cardiac pacemaker and a second leadless cardiac pacemaker, said second leadless cardiac pacemaker being intended to replace the first leadless cardiac pacemaker, wherein the computing device communicates with the first leadless cardiac pacemaker and the second leadless cardiac pacemaker by means of a wireless transmit-receive unit and comprises a user control interface.

Furthermore, the method comprises assigning a unique identifier to each of the first leadless cardiac pacemaker and the second leadless cardiac pacemaker by means of the computing device and preconfiguring the parameters of the first leadless cardiac pacemaker and/or the second leadless cardiac pacemaker by means of the computing device regardless of a location of the first leadless cardiac pacemaker and the second leadless cardiac pacemaker with respect to a transmit-receive unit of the computing device.

In addition, the method comprises setting the preconfigured parameters, when the preconfigured first leadless cardiac pacemaker and/or the preconfigured second leadless cardiac pacemaker reside in a communication range of the transmit-receive unit of the computing device.

The present invention further provides a system for programming leadless cardiac pacemakers, comprising a computing device, in particular a programmer, a first leadless cardiac pacemaker and a second leadless cardiac pacemaker, wherein the computing device is configured to set a plurality of parameters of at least the first leadless cardiac pacemaker and the second leadless cardiac pacemaker, said second leadless cardiac pacemaker being intended to replace the first leadless cardiac pacemaker, wherein the computing device is configured to communicate with the first leadless cardiac pacemaker and the second leadless cardiac pacemaker by means of a wireless transmit-receive unit and comprises a user control interface.

Furthermore, the computing device is configured to assign a unique identifier to each of the first leadless cardiac pacemaker and the second leadless cardiac pacemaker, wherein the computing device is configured to preconfigure the parameters of the first leadless cardiac pacemaker and/or the second leadless cardiac pacemaker regardless of a location of the first leadless cardiac pacemaker and the second leadless cardiac pacemaker with respect to a transmit-receive unit of the computing device, and wherein the computing device is configured to set the preconfigured parameters, when the preconfigured first leadless cardiac pacemaker and/or the preconfigured second leadless cardiac pacemaker reside in a communication range of the transmit-receive unit of the computing device.

Moreover, the present invention provides a computer-readable data carrier containing program code of a computer program for performing the method according to the present invention when the computer program is executed on a computer.

It is an idea of the present invention to treat replacement/change-out processes as a single system routine that handles the old and new device therapies in a coordinated manner that weaves their separate behaviors into a more interactive effort.

Due to limited data transmission ability of leadless cardiac pacemakers (stemming from their deeper implantation depths within patient anatomy), if two leadless cardiac pacemakers are within the range of the programmer, instead of transmitting the serial number of each device in every communication, the serial number is shortened into a unique identifier. Said unique identifier can be as simple as designating the first leadless cardiac pacemaker as device 1 and the second leadless cardiac pacemaker as device 2, wherein each of the first leadless cardiac pacemaker and the second leadless cardiac pacemaker have a serial number that is related to the shortened unique identifier.

A scheme for the wireless transmit-receive unit, i.e., the wand, to communicate with the first leadless cardiac pacemaker and the second leadless cardiac pacemaker without necessarily having to have both of the pacemakers within the range of the Programmer's communication wand at the same time is hence provided.

If the device which the user wants to program is out of the programmer's range of communication, a message will appear to the user that the programmer needs to be placed in proximity to the device that the user wants to program.

Further advantages of the present invention are access to improved robustness in the administration of bradycardia support following device replacement/change-out procedures, improved clinician control and workflows on the specifics associated with replacement/change-out that offer means to cater the go forward approach to individual patient needs, and a greater freedom to implant a replacement device long before end of service as the new device can be configured to switch on when it is needed without concern that the old device battery will not be fully utilized.

According to an aspect of the present invention, during simultaneous operation of the first leadless cardiac pacemaker and the second leadless cardiac pacemaker the first leadless cardiac pacemaker is programmed to pace at a lower than previous pacing rate while a sensing test, an impedance test and/or a pacing capture test of the second leadless cardiac pacemaker is performed.

In other words, for nominal follow-up test configurations, any pacing output from the second leadless cardiac pacemaker would be test-specific and occur temporarily while needed for test execution. Preferentially, the two approaches could be combined into a single embodiment where during test execution, the pacing occurs in accordance with test needs and at the completion of the testing the implant returns to a permanent program operation which, depending on the condition of the master therapy switch would either be administered or not.

In other words, if the master therapy switch is ON, at the end of test completion, the second leadless cardiac pacemaker would pace in accordance with its permanent program settings. If the master therapy switch is OFF, at the end of test completion, the second leadless cardiac pacemaker would halt any paced output in accordance even though it houses a permanent program with prescribed therapy settings.

According to a further aspect of the present invention, with the second leadless cardiac pacemaker pacing, the first leadless cardiac pacemaker is set to a sensing mode until battery expiration, wherein the first leadless cardiac pacemaker only resumes pacing in case of a malfunction of the second leadless cardiac pacemaker. This setting advantageously provides an additional layer of security in case of malfunction of the second leadless cardiac pacemaker.

According to a further aspect of the present invention, the first leadless cardiac pacemaker is set to a non-permanently disabled state, in which it neither senses nor paces but is still able to receive commands from the computing device and is activatable by the computing device or wherein the first leadless cardiac pacemaker is set to a permanently disabled state in which it is not activatable by the computing device.

In setting the first leadless cardiac pacemaker to either the permanently or non-permanently disabled state, it can be advantageously avoided that any non-intended pacing by the first leadless cardiac pacemaker occurs.

According to a further aspect of the present invention, a pacing program is preconfigured in the second leadless cardiac pacemaker, said preconfigured pacing program being disabled during a first predefined time period, in particular during routing into the patient, and said preconfigured pacing program being enabled during a subsequent second predefined time period, in particular when the second leadless cardiac pacemaker is anchored within the heart wall of the patient.

Thus a scheme for stationing a program within the second cardiac leadless pacemaker is provided according to which the second cardiac leadless pacemaker resides in a state where the paced output is turned off-enabling safe routing into the patient and anchoring within the heart wall without instating electrical stimulation that would incline the heart wall to recoil and/or retract.

The procedures used to physically route the second cardiac leadless pacemaker from a venous groin access occur without having the device administering stimulation output. Such an approach assists in helping the implant avoid electrically interacting with patient physiology in undesired and potentially problematic ways. This capacity especially assists in supporting implant deployment and anchoring within the heart without introducing a competing challenge associated with the initiation of action potentials and depolarization within the targeted physiologic implantation site. Such depolarization would tend to motivate contraction responses that would risk having the implantation site physically recoil/retract from the implant potentially confounding robust mechanical interfacing and/or instating unintended harm to patient physiology.

According to a further aspect of the present invention, a pacing output of the second leadless cardiac pacemaker is turned off, in particular via a master switch, through a user control interface of the computing device. This master switch setting would not however, in preferred embodiments deny the execution of sensing, impedance, and/or pacing capture threshold tests by the second leadless cardiac pacemaker. This approach nominally avoids the delivery of paced output from the device excepting conditions where such output can serve to determine the electrical characteristics at the second leadless cardiac pacemaker's anchor site. Users can thereby readily evaluate a multitude of implantation sites without having to deliberately manage the paced output settings from the second implant if it is repositioned from one anchor site to another.

Alternatively or additionally, a pacing output of the second leadless cardiac pacemaker may be turned off, in particular via a master switch, through the user control interface of the computing device, while a sensing test, an impedance test and/or a pacing capture test of the second leadless cardiac pacemaker is performed.

According to a further aspect of the present invention, the first leadless cardiac pacemaker is programmed to provide pacing output until battery expiration or until its battery voltage falls below a predetermined threshold value, at which point the first leadless cardiac pacemaker transitions to a disabled state, and wherein the second leadless cardiac pacemaker is activated if it does not sense any pacing by the first leadless cardiac pacemaker for a predetermined amount of time, in particular using a configuration set during a replacement procedure.

Thus, once battery decay below a certain limit occurs, the first leadless cardiac pacemaker locks the output to a non-therapeutic state to avoid the pacing at later points in case the battery somehow recovered to some extent.

The benefit of this extended support option is that the system can take full advantage of the available therapeutic output from the old implant before asking the new device to provide patient support. Such an approach maximizes the effective service time of any single implant and offers a best means for lowering the frequency of replacement procedures. Such a capability additional offers clinicians greater flexibilities for when the second leadless cardiac pacemaker can be implanted. Typically such procedures occur in the last 6 months of battery service for expiring implants, but given the offerings of the present invention, clinicians could implant the second device prior to the first device reaching ERI without meaningfully compromising device longevity from the second device (if doing so happened to serve a particular patient's needs). The second leadless pacer would simply reside within the patient ready to offer bradycardia symptom remediation therapy until called upon to do so subject to sufficiently degraded conditions from the first leadless pacer.

According to a further aspect of the present invention, the first leadless cardiac pacemaker provides pacing output at a rate higher than a configured pacing rate of the second leadless cardiac pacemaker, wherein the second leadless cardiac pacemaker is set to a sensing mode or effectively inhibited until battery expiration of the first leadless cardiac pacemaker or until the battery voltage of the first leadless cardiac pacemaker falls below a predetermined threshold value, at which point the first leadless cardiac pacemaker transitions to a disabled state. This way, the first leadless cardiac pacemaker is advantageously able to operate until battery expiration.

According to a further aspect of the present invention, the first leadless cardiac pacemaker encodes its pacing output during a period after a replacement procedure such that the second leadless cardiac pacemaker detects that the first leadless cardiac pacemaker provides pacing output. This approach advantageously enables the second leadless cardiac pacemaker to be aware of the first leadless cardiac pacemaker pacing.

According to a further aspect of the present invention, the first leadless cardiac pacemaker provides pacing output at a rate lower than its configured therapeutic pacing rate when its battery voltage falls below a predetermined threshold value, wherein the second leadless cardiac pacemaker detects the lowered pacing rate of the first leadless cardiac pacemaker and initiates pacing output at a higher rate than the first leadless cardiac pacemaker.

This permits the go forward therapy from the old device, i.e., the first leadless cardiac pacemaker, to adopt the intended therapeutic rate rather than one elevated to an extent to overdrive the new implant, i.e., the second leadless cardiac pacemaker. Once the new device stopped witnessing the encoded signatures on the paced output from the old device or recognized that pacing on the old device had slowed below the minimum rate, it then detects it needs to take over and provide therapy support.

According to a further aspect of the present invention, the user control interface of the computing device offers a means for clinicians to port the therapy program with which the first leadless cardiac pacemaker is operated to the second leadless cardiac pacemaker as part of in-clinic device change-out procedures.

Alternatively or additionally, a message is displayed on the user control interface of the computing device whether the therapy program with which the first leadless cardiac pacemaker is operated is to be ported to the second leadless cardiac pacemaker.

Settings from the old device are thereby transferred from the old device to the programmer. The programmer then transfers those settings to the new device upon confirmation from the user. The system thus offers a means to extract the last good viable permanent program configuration from the old device and offer a means to push/port it to the new implant as a starting setup.

According to a further aspect of the present invention, in case of a reset of the first leadless cardiac pacemaker, the first leadless cardiac pacemaker reverts to its previous therapy program and not to a default program. In particular, in case the first leadless pacemaker has been permanently disabled, after the reset the first leadless pacemaker may be configured to remain permanently disabled and does not “wake up” to deliver therapy.

The device thus remembers its previous operating state/program and does not revert to a default/factory program.

The system's reset handling can also be extended such that the old implant has access to information regarding which settings were configured by a clinician during the replacement/change-out procedure and preserves them after recovering from a reset-avoiding undesirable behaviors like post-reset competitive pacing from the old implant.

In a further advantageous embodiment of the method according to the present invention, a pacing status of the first or second leadless cardiac pacemaker may be reported via pacing status indicator in the computing device.

In a further advantageous embodiment of the present invention, fields in the computing device (programmer screen) may be pre-load such that when it is decided to program the first or second leadless cardiac pacemaker, it receives a user-configured program. In other words, all of the pre-configuring may be done on the computing device side (programmer side).

The herein described features of the computer implemented method for programming leadless cardiac pacemakers are also disclosed for the system for programming leadless cardiac pacemakers and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a flowchart of a computer implemented method for programming leadless cardiac pacemakers according to preferred embodiments of the present invention; and

FIG. 2 shows a schematic view of a system for programming leadless cardiac pacemakers according to preferred embodiments of the present invention.

### DETAILED DESCRIPTION

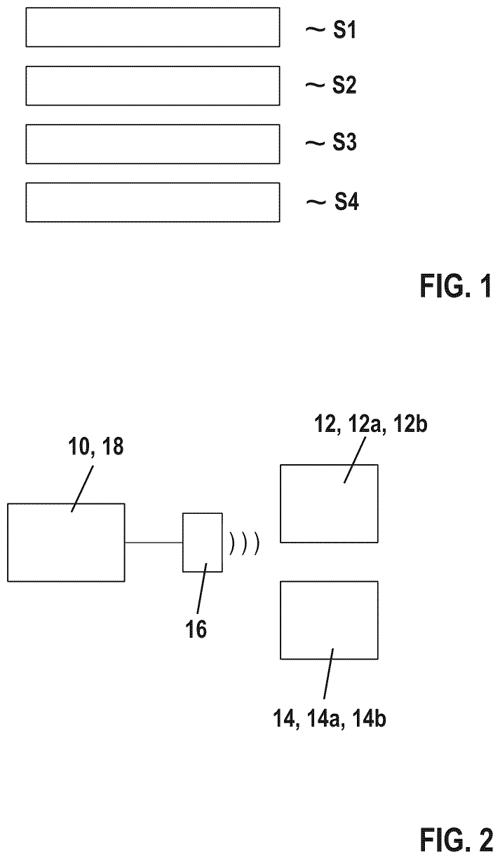

The computer implemented method of FIG. 1 serves to program leadless cardiac pacemakers 12, 14 comprising the step of providing S1 a computing device 10, in particular a programmer, configured to set a plurality of parameters 12a, 14a of at least a first leadless cardiac pacemaker 12 and a second leadless cardiac pacemaker 14, said second leadless cardiac pacemaker 14 being intended to replace the first leadless cardiac pacemaker 12, wherein the computing device 10 communicates with the first leadless cardiac pacemaker 12 and the second leadless cardiac pacemaker 14 by means of a wireless transmit-receive unit 16 and comprises a user control interface 18.

Furthermore, the method comprises the step of assigning S2 a unique identifier 12b, 14b to each of the first leadless cardiac pacemaker 12 and the second leadless cardiac pacemaker 14 by means of the computing device 10.

The method further comprises the step of preconfiguring S3 the parameters 12a, 14a of the first leadless cardiac pacemaker 12 and/or the second leadless cardiac pacemaker 14 by means of the computing device 10 regardless of a location of the first leadless cardiac pacemaker 12 and the second leadless cardiac pacemaker 14 with respect to a transmit-receive unit 16 of the computing device 10, and the step of setting S4 the preconfigured parameters 12a, 14a, when the preconfigured first leadless cardiac pacemaker 12 and/or the preconfigured second leadless cardiac pacemaker 14 reside in a communication range of the transmit-receive unit 16 of the computing device 10.

According to a preferred embodiment, during simultaneous operation of the first leadless cardiac pacemaker 12 and the second leadless cardiac pacemaker 14 the first leadless cardiac pacemaker 12 is programmed to pace at a lower than previous pacing rate while a sensing test, an impedance test and/or a pacing capture test of the second leadless cardiac pacemaker 14 is performed.

With the second leadless cardiac pacemaker 14 pacing, the first leadless cardiac pacemaker 12 is set to a sensing mode until battery expiration, wherein the first leadless cardiac pacemaker 12 only resumes pacing in case of a malfunction of the second leadless cardiac pacemaker 14.

Alternatively, with the second leadless cardiac pacemaker 14 pacing, the first leadless cardiac pacemaker 12 is effectively inhibited until battery expiration, wherein the first leadless cardiac pacemaker 12 only resumes pacing in case of a malfunction of the second leadless cardiac pacemaker 14.

According to a further preferred embodiment, the first leadless cardiac pacemaker 12 is set to a non-permanently disabled state, in which it neither senses nor paces, but is still able to receive commands from the computing device 10 and is activatable by the computing device 10 or wherein the first leadless cardiac pacemaker 12 is set to a permanently disabled state in which it is not activatable by the computing device 10.

A pacing program is preconfigured in the second leadless cardiac pacemaker 14, said preconfigured pacing program being disabled during a first predefined time period, in particular during routing into a patient, and said preconfigured pacing program being enabled during a subsequent second predefined time period, in particular when the second leadless cardiac pacemaker 14 is anchored within the heart wall of the patient, or in particular when the second leadless cardiac pacemaker 14 is viably anchored within the heart wall of the patient at its go-forward final implantation site.

A pacing output of the second leadless cardiac pacemaker 14 is turned off, in particular via a master switch, through the user control interface 18 of the computing device 10, while a sensing test, an impedance test and/or a pacing capture test of the second leadless cardiac pacemaker 14 is performed-nominally disabling pace output from the second device barring output affiliated with testing; such methods supporting the ability to probe a multitude of candidate anchoring sites before configuring the second implant with a go-forward permanent program therapy output condition.

According to a further preferred embodiment, the first leadless cardiac pacemaker 12 is programmed to provide pacing output until battery expiration or until its battery voltage falls below a predetermined threshold value, at which point the first leadless cardiac pacemaker 12 transitions to a disabled state. Moreover, the second leadless cardiac pacemaker 14 is activated if it does not sense any pacing by the first leadless cardiac pacemaker 12 for a predetermined amount of time, in particular using a configuration set during a replacement procedure.

According to a further preferred embodiment, the first leadless cardiac pacemaker 12 provides pacing output at a rate higher than a configured pacing rate of the second leadless cardiac pacemaker 14, wherein the second leadless cardiac pacemaker 14 is inhibited until battery expiration of the first leadless cardiac pacemaker 12 or until the battery voltage of the first leadless cardiac pacemaker 12 falls below a predetermined threshold value, at which point the first leadless cardiac pacemaker 12 transitions to a disabled state.

The first leadless cardiac pacemaker 12 encodes its pacing output during a period after a replacement procedure such that the second leadless cardiac pacemaker 14 detects that the first leadless cardiac pacemaker 12 provides pacing output.

Furthermore, the first leadless cardiac pacemaker 12 provides pacing output at a rate lower than its configured therapeutic pacing rate when its battery voltage falls below a predetermined threshold value, wherein the second leadless cardiac pacemaker 14 detects the lowered pacing rate of the first leadless cardiac pacemaker 12 and initiates pacing output at a higher rate than the first leadless cardiac pacemaker 12.

The user control interface 18 of the computing device 10 offers a means for clinicians to port the therapy program with which the first leadless cardiac pacemaker 12 is operated to the second leadless cardiac pacemaker 14 as part of in-clinic device change-out procedures. In case of a reset of the first leadless cardiac pacemaker 12, the first leadless cardiac pacemaker 12 reverts to its previous therapy program and not to a default program.

FIG. 2 shows a schematic view of a system for programming leadless cardiac pacemakers according to preferred embodiments of the present invention.

The system 1 for programming leadless cardiac pacemakers comprises a computing device 10, in particular a programmer, a first leadless cardiac pacemaker 12 and a second leadless cardiac pacemaker 14.

The computing device 10 is configured to set a plurality of parameters 12a, 14a of at least the first leadless cardiac pacemaker 12 and the second leadless cardiac pacemaker 14, said second leadless cardiac pacemaker 14 being intended to replace the first leadless cardiac pacemaker 12. Furthermore, the computing device 10 is configured to communicate with the first leadless cardiac pacemaker 12 and the second leadless cardiac pacemaker 14 by means of a wireless transmit-receive unit 16 and comprises a user control interface 18.

Moreover, the computing device 10 is configured to assign a unique identifier 12b, 14b to each of the first leadless cardiac pacemaker 12 and the second leadless cardiac pacemaker 14. The computing device 10 is further configured to preconfigure the parameters 12a, 14a of the first leadless cardiac pacemaker 12 and/or the second leadless cardiac pacemaker 14 regardless of a location of the first leadless cardiac pacemaker 12 and the second leadless cardiac pacemaker 14 with respect to a transmit-receive unit 16 of the computing device 10. In addition, the computing device 10 is configured to set the preconfigured parameters 12a, 14a, when the preconfigured first leadless cardiac pacemaker 12 and/or the preconfigured second leadless cardiac pacemaker 14 reside in a communication range of the transmit-receive unit 16 of the computing device 10.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 System
10 computing device
12 first leadless cardiac pacemaker
12a parameters
12b unique identifier
14 second leadless cardiac pacemaker
14a parameters
14b unique identifier
16 transmit-receive unit
18 user control interface
S1-S4 method steps

1. Computer implemented method for programming leadless cardiac pacemakers comprising the steps of:
providing a computing device, in particular a programmer, configured to set a plurality of parameters of at least a first leadless cardiac pacemaker and a second leadless cardiac pacemaker, said second leadless cardiac pacemaker being intended to replace the first leadless cardiac pacemaker, wherein the computing device communicates with the first leadless cardiac pacemaker and the second leadless cardiac pacemaker by means of a wireless transmit-receive unit and comprises a user control interface;
assigning a unique identifier to each of the first leadless cardiac pacemaker and the second leadless cardiac pacemaker by means of the computing device;
reconfiguring the parameters of the first leadless cardiac pacemaker and/or the second leadless cardiac pacemaker by means of the computing device regardless of a location of the first leadless cardiac pacemaker and the second leadless cardiac pacemaker with respect to a transmit-receive unit of the computing device; and
setting the preconfigured parameters, when the preconfigured first leadless cardiac pacemaker and/or the preconfigured second leadless cardiac pacemaker reside in a communication range of the transmit-receive unit of the computing device.

2. Computer implemented method of claim 1, wherein during simultaneous operation of the first leadless cardiac pacemaker and the second leadless cardiac pacemaker the first leadless cardiac pacemaker is programmed to pace at a lower than previous pacing rate while a sensing test, an impedance test and/or a pacing capture test of the second leadless cardiac pacemaker is performed.

3. Computer implemented method of claim 1, wherein with the second leadless cardiac pacemaker pacing, the first leadless cardiac pacemaker is set to a sensing mode or effectively inhibited until battery expiration, wherein the first leadless cardiac pacemaker only resumes pacing in case of a malfunction of the second leadless cardiac pacemaker.

4. Computer implemented method of claim 1, wherein the first leadless cardiac pacemaker is set to a non-permanently disabled state, in which it neither senses nor paces, but is still able to receive commands from the computing device and is activatable by the computing device or wherein the first leadless cardiac pacemaker is set to a permanently disabled state in which it is not activatable by the computing device.

5. Computer implemented method of claim 1, wherein a pacing program is preconfigured in the second leadless cardiac pacemaker, said preconfigured pacing program being disabled during a first predefined time period, in particular during routing into a patient, and said preconfigured pacing program being enabled during a subsequent second predefined time period, in particular when the second leadless cardiac pacemaker is anchored within the heart wall of the patient.

6. Computer implemented method of claim 1, wherein a pacing output of the second leadless cardiac pacemaker is turned off, in particular via a master switch, through the user control interface of the computing device.

7. Computer implemented method of claim 1, wherein the first leadless cardiac pacemaker is programmed to provide pacing output until battery expiration or until its battery voltage falls below a predetermined threshold value, at which point the first leadless cardiac pacemaker transitions to a disabled state, and wherein the second leadless cardiac pacemaker is activated if it does not sense any pacing by the first leadless cardiac pacemaker for a predetermined amount of time, in particular using a configuration set during a replacement procedure.

8. Computer implemented method of claim 1, wherein the first leadless cardiac pacemaker provides pacing output at a rate higher than a configured pacing rate of the second leadless cardiac pacemaker, wherein the second leadless cardiac pacemaker is set to a sensing mode or effectively inhibited until battery expiration of the first leadless cardiac pacemaker or until the battery voltage of the first leadless cardiac pacemaker falls below a predetermined threshold value, at which point the first leadless cardiac pacemaker transitions to a disabled state.

9. Computer implemented method of claim 1, wherein the first leadless cardiac pacemaker encodes its pacing output during a period after a replacement procedure such that the second leadless cardiac pacemaker detects that the first leadless cardiac pacemaker provides pacing output.

10. Computer implemented method of claim 1, wherein the first leadless cardiac pacemaker provides pacing output at a rate lower than its configured therapeutic pacing rate when its battery voltage falls below a predetermined threshold value, wherein the second leadless cardiac pacemaker detects the lowered pacing rate of the first leadless cardiac pacemaker and initiates pacing output at a higher rate than the first leadless cardiac pacemaker.

11. Computer implemented method of claim 1, wherein a message is displayed on the user control interface of the computing device whether the therapy program with which the first leadless cardiac pacemaker is operated is to be ported to the second leadless cardiac pacemaker.

12. Computer implemented method of claim 1, wherein in case of a reset of the first leadless cardiac pacemaker, the first leadless cardiac pacemaker reverts to its previous therapy program and not to a default program.

13. System for programming leadless cardiac pacemakers, comprising a computing device, in particular a programmer, a first leadless cardiac pacemaker and a second leadless cardiac pacemaker, wherein the computing device is configured to set a plurality of parameters of at least the first leadless cardiac pacemaker and the second leadless cardiac pacemaker, said second leadless cardiac pacemaker being intended to replace the first leadless cardiac pacemaker, wherein the computing device is configured to communicate with the first leadless cardiac pacemaker and the second leadless cardiac pacemaker by means of a wireless transmit-receive unit and comprises a user control interface, wherein the computing device is configured to assign a unique identifier to each of the first leadless cardiac pacemaker and the second leadless cardiac pacemaker, wherein the computing device is configured to preconfigure the parameters of the first leadless cardiac pacemaker and/or the second leadless cardiac pacemaker regardless of a location of the first leadless cardiac pacemaker and the second leadless cardiac pacemaker with respect to a transmit-receive unit of the computing device, and wherein the computing device is configured to set the preconfigured parameters, when the preconfigured first leadless cardiac pacemaker and/or the preconfigured second leadless cardiac pacemaker reside in a communication range of the transmit-receive unit of the computing device.

14. Computer program with program code to perform the method of claim 1 when the computer program is executed on a computer.

15. Computer-readable data carrier containing program code of computer program for performing the method of claim 1 when the computer program is executed on a computer.
